(19) United States
(12) Patent Application Publication
(10) Pub. No.: US 2026/0117172 A1
(43) Pub. Date: Apr. 30, 2026

(54) Selective Sperm Retrieval System for Improved In Vitro Fertilization

(71) Applicant: JOHN GREGORY BAKER, PLANO, TX (US)

(72) Inventor: JOHN GREGORY BAKER, PLANO, TX (US)

(21) Appl. No.: 18/932,815

(22) Filed: Oct. 31, 2024

(51) Int. Cl. C12N 5/071

(52) U.S. Cl. CPC C12N 5/0612

(57) ABSTRACT

A system and method for improving sperm selection during in vitro fertilization (IVF) procedures includes a sperm containment apparatus equipped with a capture system designed to isolate motile sperm as they respond to the presence of a retrieval instrument within the sperm solution. As more motile and potentially healthier sperm move away from the retrieval instrument, they are selectively corralled within certain regions of the apparatus. The capture mechanism may include various structures, such as a barrier member including one or more passageways which direct sperm movement. By isolating sperm based on their motility, this invention enhances the selection process, improving the chances of successful fertilization with higher-quality sperm. This system provides a more efficient and targeted approach to sperm retrieval compared to conventional methods.

### BACKGROUND OF THE INVENTION

In the field of in vitro fertilization (IVF), sperm selection plays a critical role in the success of fertilization and the overall health of the embryo. Traditional techniques involve aspirating sperm from a solution using a micropipette or similar device. However, during the retrieval process, the healthier, more motile sperm often exhibit an escape response, swimming away from the probe as if reacting to an external threat. This behavior can make it challenging to isolate the highest-quality sperm, particularly in environments where expedience may lead to the selection of less vigorous sperm. Due to time constraints for the technical staff performing the sperm selection, hurried manual selection can inadvertently favor less motile sperm, potentially leading to suboptimal fertilization outcomes. The present invention provides an automated solution that minimizes the risk of such occurrences, ensuring that sperm with higher motility and viability are more consistently selected.

### FIELD OF THE INVENTION

The present invention relates to the field of assisted reproductive technologies, specifically in vitro fertilization (IVF). More particularly, it pertains to a system and method for selectively capturing motile sperm within a sperm-containing solution during the retrieval process. The invention involves the use of a trap door/baffle mechanism within a petri dish or container, designed to isolate faster, healthier sperm as they exhibit evasive movement in response to a retrieval instrument. This system aims to improve the overall success rate of fertilization by enhancing the selection of higher-quality sperm for use in IVF procedures.

### BENEFITS OF THE INVENTION

The present invention offers several significant benefits in the context of in vitro fertilization (IVF) procedures:

Improved Sperm Selection: By utilizing a trap door mechanism that selectively captures faster, more motile sperm, the invention ensures that only the most viable and healthier sperm are chosen for fertilization. This increases the chances of successful fertilization and healthy embryo development.

Enhanced Fertilization Outcomes: Since sperm motility is often correlated with sperm health and viability, the ability to isolate more motile sperm improves the likelihood of a successful fertilization process, leading to better overall reproductive outcomes for patients undergoing IVF.

Minimizes Human Error: The invention reduces the likelihood of selecting less viable sperm due to convenience or expediency, as it automates the process of isolating the best sperm, reducing the subjectivity and variability associated with manual sperm selection by embryologists.

Compatibility with Existing IVF Systems: The trap door mechanism can be easily integrated into standard petri dishes and sperm containers used in IVF laboratories, making it a seamless addition to current IVF workflows without the need for extensive changes or additional training.

Efficient Sperm Retrieval: By taking advantage of the natural behavior of motile sperm to evade disturbance, the system provides a more efficient and effective method for collecting high-quality sperm, reducing the time required for sperm selection.

Potential for Higher Pregnancy Success Rates: With more careful selection of healthier sperm, the invention may contribute to higher pregnancy success rates and lower risks of complications, enhancing the overall effectiveness of IVF treatments for patients.

These benefits position the invention as an innovative advancement in the field of reproductive technology, providing both practitioners and patients with a more reliable and efficient method for improving fertilization outcomes.

### SUMMARY OF THE INVENTION

The present invention provides a solution to the challenge of sperm selection by introducing a novel capture apparatus such as a trap door mechanism within the sperm container or petri dish. This apparatus provides an obstacle course for spermatozoa in order to separate slow moving and presumable less fit sperm from more energetic forms. When a retrieval instrument penetrates the fluid medium in the petri dish, faster-moving sperm instinctively attempt to flee the probe to be captured in a separate chamber. This trap door system captures the sperm exhibiting greater motility, which is often associated with better health and viability. By isolating these sperm, the invention ensures that only the most suitable sperm are used in fertilization, increasing the likelihood of successful implantation and healthy embryo development.

### BRIEF DESCRIPTION OF THE DRAWING FIGURES

FIG. 1 is a top perspective view of a barrier member integrated into a petri dish;

FIG. 2 is an underside view of (FIG. 1);

FIG. 3 is a to plan view of a barrier member isolated from a petri dish;

FIG. 4 is a side elevation thereof;

FIG. 5 is a top perspective view thereof;

FIG. 6 is a top perspective view of another implementation of a barrier member;

FIG. 7 is an underside view thereof;

FIG. 8 is a bottom plan view of the barrier member of (FIG. 6);

### REFERENCE LISTING OF THE NAMED ELEMENTS

(10) container or petri dish (for holding sperm solution)
(12) fluid medium (contained in the dish)
(13) spermatozoa
(14) retrieval instrument (the probe or suction device used to retrieve sperm)
(17) barrier member
(18) center surround
(19) concentric surround
(20) opening
(21) chutes
(22) (23) selective closure, magnetically responsive element
(24) corral/trap/enclosure

### DETAILED DESCRIPTION

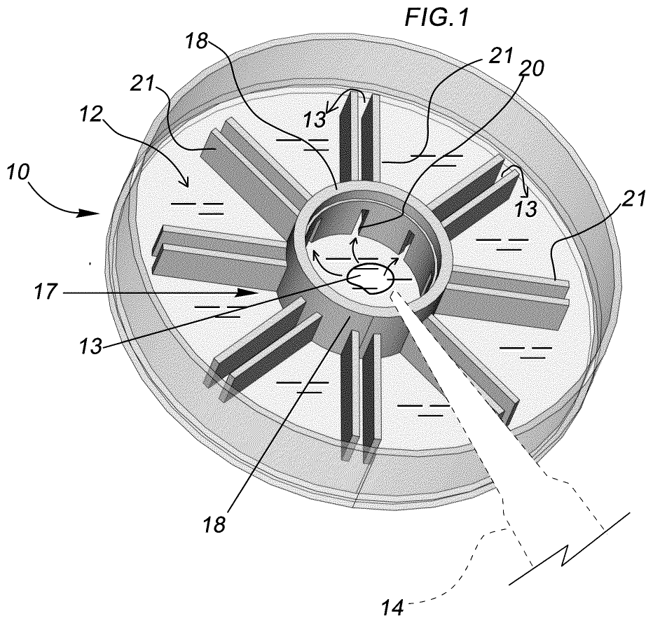

FIG. 1 illustrates a top view of the sperm selection apparatus. Container (10) is filled with a fluid medium (12), to contain the motile sperm. Positioned in or integrated with the container is a barrier member (17), that includes one or more passageways in order to direct sperm movement when the sperm are disturbed/scattered by a retrieval instrument (RI) (14). As the RI moves through the solution, the more motile sperm react by swimming away from the RI. In the implementation shown in FIG. 1, for example, a portion of a sperm collection is placed in the center surround (18) of the barrier member (17) and prompted by the RI to scatter to the border of the center surround. Various apertures or openings (20) are situated in the side(s) of the surround and provide a means of egress for individual spermatozoa. The more energetic of the spermatozoa find their way through the openings (20) and into chutes (21). The speedier of the spermatozoa find their way to the ends of the chutes faster and may be collected there by the retrieval instrument. While the chutes (21) shown are open at their terminus, in some cases there may be a molded enclosure at the terminus to collect and contain the spermatozoa. In some implementations, the barrier member may be molded as part of a plastic petri dish while in other implementations, the barrier member may be added to a conventional petri dish.

In some implementations, openings (20) may be fitted with a selective closure that may be magnetically actuated (closed or opened) by tapping the surround with a magnet tipped stylus, while in other implementations, the openings are simply formed apertures.

The various implementations described herein may be made of any material that would be appreciated by those having skill in the art that is non-toxic for the spermatozoa and includes a smooth surface to resist fluidic cohesion (clumping and drag) by the fluid medium.

In any of the various implementations described herein, the number of openings in the surround(s) can vary and the number of chutes can vary, the specific implementations shown being merely exemplary.

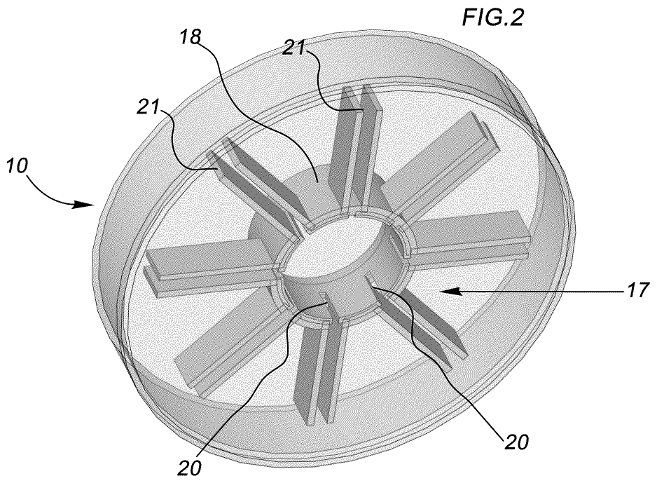

FIG. 2 is a bottom perspective view of the sperm selection apparatus of (FIG. 1). It should be noted that the openings in the center surround (18) and the chutes (21) have floors that are substantially co-planar with the petri dish bottom with ends of the chutes terminating at the periphery of the petri dish. The chutes include walls that line a passageway and the walls may be any height or thickness as would be found desirable by persons having skill in the art.

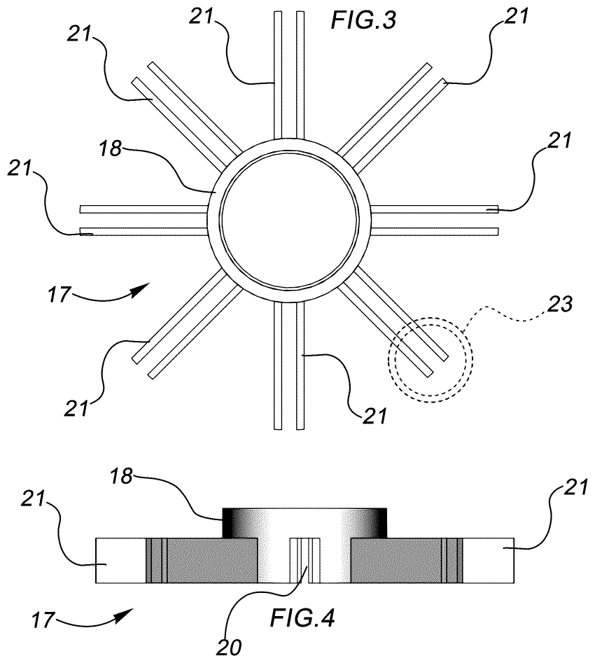

FIG. 3 shows a top plan view of one implementation of a barrier member that includes a central surround (18) with radially oriented chutes (21). In some implementations, as shown in dashed line one or more ends of the chutes may includes an enclosure (24) to trap or corral spermatozoa.

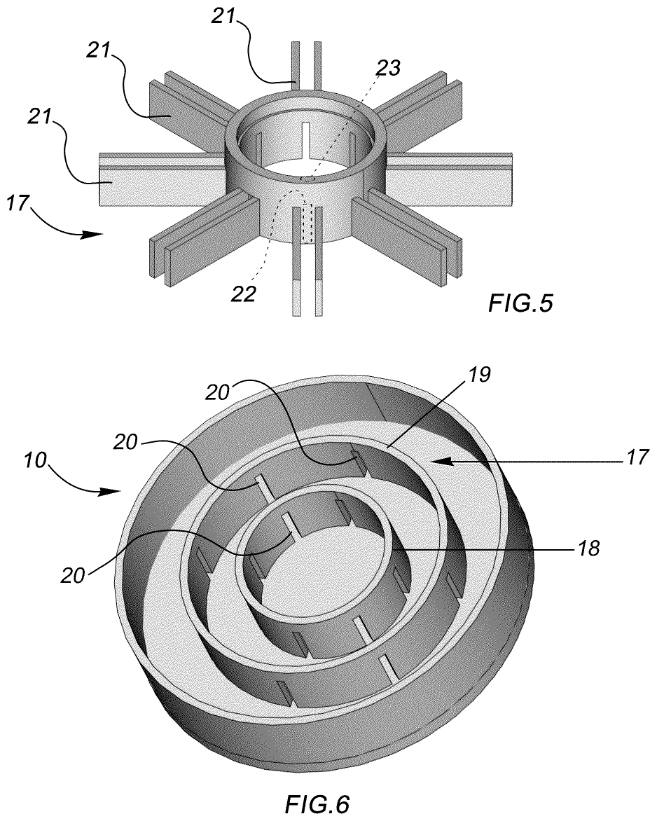

FIGS. 4 and 5 are respectively, a side elevation of the barrier member (17) shown in (FIG. 3) and a top perspective view thereof. Dashed line (22) in FIG. 5 represents an opening that has been closed. Closure mechanisms for openings (20) may be mechanical such as a sliding panel that may be depressed by a clinician, the panel sliding in front of an opening or wherein the sliding panel resides in the wall of the central surround (18) or any other portion of the barrier member. Alternately, an element may be removed or inserted by a clinician to block an opening (20) or a panel (22) may be magnetically actuated or the panel may be magnetically responsive. For example, a small magnetically responsive object (23) may reside atop all or some of the openings, its purpose to retain a closure panel in an open position such that when the magnetically responsive object is removed, the panel no longer suspended, drops to close the opening. In some implementations, the closure of an opening may be irreversible or reversible.

FIG. 6 is a top perspective view of another implementation including a relatively larger concentric surround (19) outside of the central surround (18). each including openings (20) such that sperm collection begins in the central surround and the some sperm find their way past the openings successively to the outer bounds of the petri-dish.

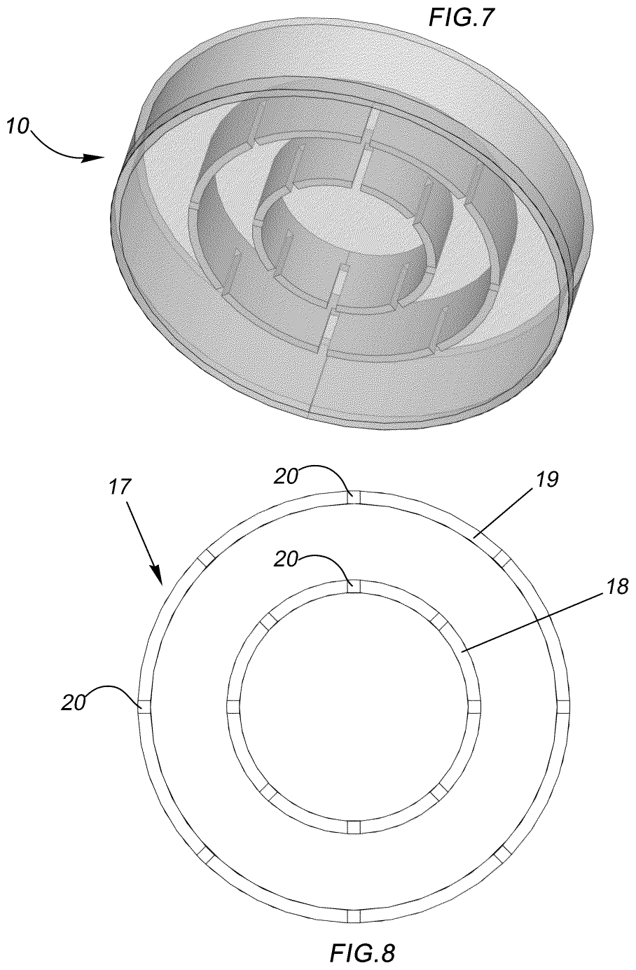

FIG. 7 is a bottom perspective view thereof and FIG. 8 is a bottom plan view thereof.

It should be understood that the drawings and detailed description herein are to be regarded in an illustrative rather than a restrictive manner, and are not intended to be limiting to the particular forms and examples disclosed. Accordingly, it is intended that this disclosure encompass any further modifications, changes, rearrangements, substitutions, alternatives, design choices, and embodiments as would be appreciated by those of ordinary skill in the art having benefit of this disclosure, and falling within the spirit and scope of the following claims.

What is claimed is:

1. A sperm selection apparatus for use in in vitro fertilization (IVF), comprising:
a container configured to hold a sperm solution;
a capture mechanism positioned within the container, wherein the capture mechanism is operable to isolate motile sperm.

2. The sperm selection apparatus according to claim 1, the capture mechanism includes one or more sperm pathways.

3. The sperm selection apparatus according to claim 1, the capture mechanism including one or more physical barriers.

4. The sperm selection apparatus according to claim 1, the capture mechanism including one or more regions with connecting passageways therebetween.

5. The sperm selection apparatus according to claim 4, the one or more connecting passageways are selectively opened.

6. A method of selecting sperm for use in in vitro fertilization (IVF), comprising:
placing a sperm solution in a container;
inserting a retrieval instrument (RI) into the sperm and prompting the sperm to move away from the RI;
driving the sperm through one or more passageways;
collecting desired sperm within or at the end of the one or more passageways.

7. The method according to claim 6, wherein some of the one or more passageways may be selectively opened or closed.
